(19) United States
(12) Patent Application Publication
(10) Pub. No.: US 2014/0180141 A1
(43) Pub. Date: Jun. 26, 2014

(54) Mounting Structures for Components of Intravascular Devices

(71) Applicant: Volcano Corporation, San Diego, CA (US)

(72) Inventor: Bret C. Millett, Folsom, CA (US)

(73) Assignee: Volcano Corporation, San Diego, CA (US)

(21) Appl. No.: 14/135,117

(22) Filed: Dec. 19, 2013

Related U.S. Application Data
(60) Provisional application No. 61/745,467, filed on Dec. 21, 2012.

(51) Int. Cl. A61B 5/0215; A61M 25/00; A61M 25/09

(52) U.S. Cl. CPC A61B 5/0215; A61M 25/09; A61M 25/0009
USPC 600/486; 29/857

(57) ABSTRACT

Intravascular devices, systems, and methods are disclosed. In some embodiments, the intravascular devices include at least one mounting structure within a distal portion of the device. In that regard, one or more electronic, optical, and/or electro-optical component is coupled to the mounting structure. Methods of making and/or assembling such intravascular devices/systems are also provided.

### CROSS-REFERENCE TO RELATED APPLICATIONS

The present application claims priority to and the benefit of U.S. Provisional Patent Application No. 61/745,467, filed Dec. 21, 2012, which is hereby incorporated by reference in its entirety.

### TECHNICAL FIELD

The present disclosure relates to intravascular devices, systems, and methods. In some embodiments, the intravascular devices are guide wires that include a mounting structure for one or more sensing components.

### BACKGROUND

Heart disease is very serious and often requires emergency operations to save lives. A main cause of heart disease is the accumulation of plaque inside the blood vessels, which eventually occludes the blood vessels. Common treatment options available to open up the occluded vessel include balloon angioplasty, rotational atherectomy, and intravascular stents. Traditionally, surgeons have relied on X-ray fluoroscopic images that are planar images showing the external shape of the silhouette of the lumen of blood vessels to guide treatment. Unfortunately, with X-ray fluoroscopic images, there is a great deal of uncertainty about the exact extent and orientation of the stenosis responsible for the occlusion, making it difficult to find the exact location of the stenosis. In addition, though it is known that restenosis can occur at the same place, it is difficult to check the condition inside the vessels after surgery with X-ray.

A currently accepted technique for assessing the severity of a stenosis in a blood vessel, including ischemia causing lesions, is fractional flow reserve (FFR). FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment.

Often intravascular catheters and guide wires are utilized to measure the pressure within the blood vessel, visualize the inner lumen of the blood vessel, and/or otherwise obtain data related to the blood vessel. To date, guide wires containing pressure sensors, imaging elements, and/or other electronic, optical, or electro-optical components have suffered from reduced performance characteristics compared to standard guide wires that do not contain such components. For example, the handling performance of previous guide wires containing electronic components have been hampered, in some instances, by the limited space available for the core wire after accounting for the space needed for the conductors or communication lines of the electronic component(s), the stiffness and size of the rigid housing containing the electronic component(s), and/or other limitations associated with providing the functionality of the electronic components in the limited space available within a guide wire.

Accordingly, there remains a need for improved intravascular devices, systems, and methods that include a mounting structure for one or more electronic, optical, or electro-optical sensing components.

### SUMMARY

Embodiments of the present disclosure are directed to intravascular devices, systems, and methods.

In one embodiment, a guide wire is provided. The guide wire comprises: a first flexible element; a second flexible element; a mounting structure coupled to the first and second flexible elements such that a central portion of the mounting structure separates the first flexible element from the second flexible element, the mounting structure comprising a recess within an outer surface, the recess sized and shaped to receive a pressure sensing component; a pressure sensing component mounted within the recess of the mounting structure; a core extending along a length of the mounting structure such that a first portion of the core is positioned within the first flexible element and a second portion of the core is positioned within the second flexible element; and at least one conductor having a proximal section and a distal section, wherein the distal section of the at least one conductor is coupled to the pressure sensing component and the proximal section of the at least one conductor is coupled to at least one connector;

In some instances, the first flexible element, the second flexible element, and the mounting structure each have an outer diameter of 0.018″ or less, such as 0.014″ or less. In some implementations, the mounting structure further comprises an opening extending along its length and the core is positioned within the opening. In some instances, a first portion of the mounting structure and a first portion of the core define a first alignment feature sized and shaped to align engagement of the first flexible element with the mounting structure. The first alignment feature may have circular cross-sectional profile such that a section of an outer surface of the first portion of the core defines at least a portion of the circular cross-sectional profile of the first alignment feature. Further, the first alignment feature may have a cross-sectional diameter less than a cross-sectional diameter of the central portion of the mounting structure. In some instances, a second portion of the mounting structure and a second portion of the core define a second alignment feature sized and shaped to align engagement of the second flexible element with the mounting structure. In some embodiments, the opening is sized and shaped such that the core received within the opening is coaxial with respect to a central longitudinal axis of the mounting structure. In other embodiments, the opening is sized and shaped such that the core received within the opening is radially offset with respect to a central longitudinal axis of the mounting structure. In that regard, the opening is radially offset in a direction away from the recess of the mounting structure in some instances. In some implementations, the opening of the mounting structure is spaced from outer surfaces of the mounting structure such that mounting structure surrounds the core positioned within the opening.

In another embodiment, a method of assembling a guide wire is provided. The method includes: providing a core wire with a flattened section; securing a mounting structure to the flattened section of the core wire, the mounting structure comprising a recess within an outer surface, the recess sized and shaped to receive a pressure sensing component; securing a pressure sensing component within the recess of the mounting structure, the pressure sensing component electrically coupled to a plurality of conductors; securing a first flexible element to a proximal portion of the mounting structure; securing a second flexible element to a distal portion of the mounting structure such that a section of the second flexible element extends over a pressure sensitive region of the pressure sensing component; and electrically coupling the plurality of conductors to a connector adjacent a proximal portion of the core wire.

In some instances, the first flexible element, the second flexible element, and the mounting structure each have an outer diameter of 0.018″ or less, such as 0.014″ or less. In some instances, a first portion of the mounting structure and a first portion of the core define a first alignment feature sized and shaped to align engagement of the first flexible element with the mounting structure. The first alignment feature may have circular cross-sectional profile such that a section of an outer surface of the first portion of the core defines at least a portion of the circular cross-sectional profile of the first alignment feature. Further, the first alignment feature may have a cross-sectional diameter less than a cross-sectional diameter of the central portion of the mounting structure. In some instances, a second portion of the mounting structure and a second portion of the core define a second alignment feature sized and shaped to align engagement of the second flexible element with the mounting structure.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic, schematic side view of an intravascular device according to an embodiment of the present disclosure.

FIG. 2 is a diagrammatic cross-sectional side view of an intravascular device according to an embodiment of the present disclosure.

FIG. 3 is a diagrammatic perspective view of a distal portion of an intravascular device including a mounting structure according to an embodiment of the present disclosure.

FIG. 4 is a perspective view of a partially assembled distal portion of an intravascular device including a mounting structure with a pressure sensor mounted in a face down configuration according to an embodiment of the present disclosure.

FIG. 5 is a perspective view of a partially assembled distal portion of an intravascular device including a mounting structure with a pressure sensor mounted in a face up configuration according to an embodiment of the present disclosure.

FIG. 6 is a diagrammatic end view of a mounting structure coupled with a core according to an embodiment of the present disclosure.

FIG. 7 is a diagrammatic perspective bottom view of the mounting structure and core of FIG. 6.

FIG. 8 is a diagrammatic perspective view of a mounting structure coupled with a core according to an embodiment of the present disclosure.

FIG. 9 is a perspective view of a mounting structure coupled with a core according to an embodiment of the present disclosure.

FIG. 10 is a perspective view of the mounting structure and core of FIG. 9, shown with a sensing element and communications lines coupled to the mounting structure such that the sensing element is in a face down configuration.

FIG. 11 is a perspective view of the mounting structure and core of FIG. 9, shown with a sensing element and communications lines coupled to the mounting structure such that the sensing element is in a face up configuration.

FIG. 12 is a perspective view of a distal portion of a core wire according to an embodiment of the present disclosure.

FIG. 13 is a perspective view of a section of the distal portion of the core wire of FIG. 12 according to an embodiment of the present disclosure.

FIG. 14 is a perspective view of a mounting structure secured to the distal portion of the core wire of FIGS. 12 and 13.

FIG. 15 is a perspective view of a pressure sensor and a plurality of conductors electrically coupled to the pressure sensor according to an embodiment of the present disclosure.

FIG. 16 is a perspective view of an adhesive being applied to surfaces of the mounting structure of FIG. 14 according to an embodiment of the present disclosure.

FIG. 17 is a perspective view of the pressure sensor and plurality of conductors of FIG. 15 mounted to the mounting structure by the adhesive of FIG. 16 according to an embodiment of the present disclosure.

FIG. 18 is a perspective view of a proximal coil being positioned adjacent to a proximal end portion of the mounting structure.

FIG. 19 is a perspective view of the proximal coil being secured to the proximal end portion of the mounting structure with an adhesive.

FIG. 20 is a perspective view of a distal coil being positioned adjacent to a distal end portion of the mounting structure.

FIG. 21 is a perspective view of the distal coil being secured to the distal end portion of the mounting structure with an adhesive.

FIG. 22 is a side view of the distal coil secured to the mounting structure.

FIG. 23 is a cross-sectional side view of the distal coil secured to the mounting structure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guide wires and catheters. In that regard, catheters may or may not include a lumen extending along its length for receiving and/or guiding other instruments. If the catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a temperature sensor, an imaging element, an optical fiber, an ultrasound transducer, a reflector, a minor, a prism, an ablation element, an RF electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized.

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the elongate member, such as a guide wire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm) and approximately 0.018″ (0.4572 mm)). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

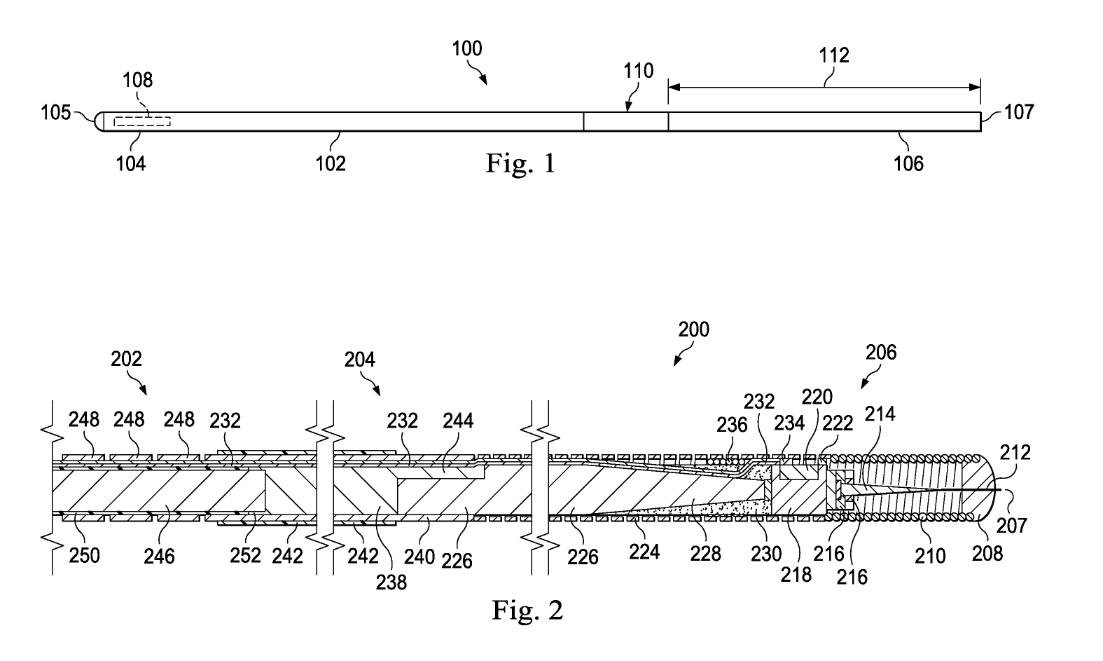

Referring now to FIG. 1, shown therein is a portion of an intravascular device 100 according to an embodiment of the present disclosure. In that regard, the intravascular device 100 includes a flexible elongate member 102 having a distal portion 104 adjacent a distal end 105 and a proximal portion 106 adjacent a proximal end 107. A component 108 is positioned within the distal portion 104 of the flexible elongate member 102 proximal of the distal tip 105. Generally, the component 108 is representative of one or more electronic, optical, or electro-optical components. In that regard, the component 108 is a pressure sensor, a temperature sensor, an imaging element, an optical fiber, an ultrasound transducer, a reflector, a minor, a prism, an ablation element, an RF electrode, a conductor, and/or combinations thereof. The specific type of component or combination of components can be selected based on an intended use of the intravascular device. In some instances, the component 108 is positioned less than 10 cm, less than 5, or less than 3 cm from the distal tip 105. In some instances, the component 108 is positioned within a housing of the flexible elongate member 102. In that regard, the housing is a separate component secured to the flexible elongate member 102 in some instances. In other instances, the housing is integrally formed as a part of the flexible elongate member 102.

The intravascular device 100 also includes a connector 110 adjacent the proximal portion 106 of the device. In that regard, the connector 110 is spaced from the proximal end 107 of the flexible elongate member 102 by a distance 112. Generally, the distance 112 is between 0% and 50% of the total length of the flexible elongate member 102. While the total length of the flexible elongate member can be any length, in some embodiments the total length is between about 1300 mm and about 4000 mm, with some specific embodiments have a length of 1400 mm, 1900 mm, and 3000 mm. Accordingly, in some instances the connector 110 is positioned at the proximal end 107. In other instances, the connector 110 is spaced from the proximal end 107. For example, in some instances the connector 110 is spaced from the proximal end 107 between about 0 mm and about 1400 mm. In some specific embodiments, the connector 110 is spaced from the proximal end by a distance of 0 mm, 300 mm, and 1400 mm.

The connector 110 is configured to facilitate communication between the intravascular device 100 and another device. More specifically, in some embodiments the connector 110 is configured to facilitate communication of data obtained by the component 108 to another device, such as a computing device or processor. Accordingly, in some embodiments the connector 110 is an electrical connector. In such instances, the connector 110 provides an electrical connection to one or more electrical conductors that extend along the length of the flexible elongate member 102 and are electrically coupled to the component 108. In other embodiments, the connector 110 is an optical connector. In such instances, the connector 110 provides an optical connection to one or more optical communication pathways (e.g., fiber optic cable) that extend along the length of the flexible elongate member 102 and are optically coupled to the component 108. Further, in some embodiments the connector 110 provides both electrical and optical connections to both electrical conductor(s) and optical communication pathway(s) coupled to the component 108. In that regard, it should again be noted that component 108 is comprised of a plurality of elements in some instances. In some instances, the connector 110 is configured to provide a physical connection to another device, either directly or indirectly. In other instances, the connector 110 is configured to facilitate wireless communication between the intravascular device 100 and another device. Generally, any current or future developed wireless protocol(s) may be utilized. In yet other instances, the connector 110 facilitates both physical and wireless connection to another device.

As noted above, in some instances the connector 110 provides a connection between the component 108 of the intravascular device 100 and an external device. Accordingly, in some embodiments one or more electrical conductors, one or more optical pathways, and/or combinations thereof extend along the length of the flexible elongate member 102 between the connector 110 and the component 108 to facilitate communication between the connector 110 and the component 108. Generally, any number of electrical conductors, optical pathways, and/or combinations thereof can extend along the length of the flexible elongate member 102 between the connector 110 and the component 108. In some instances, between one and ten electrical conductors and/or optical pathways extend along the length of the flexible elongate member 102 between the connector 110 and the component 108. For the sake of clarity and simplicity, the embodiments of the present disclosure described below include three electrical conductors. However, it is understood that the total number of communication pathways and/or the number of electrical conductors and/or optical pathways is different in other embodiments. More specifically, the number of communication pathways and the number of electrical conductors and optical pathways extending along the length of the flexible elongate member 102 is determined by the desired functionality of the component 108 and the corresponding elements that define component 108 to provide such functionality.

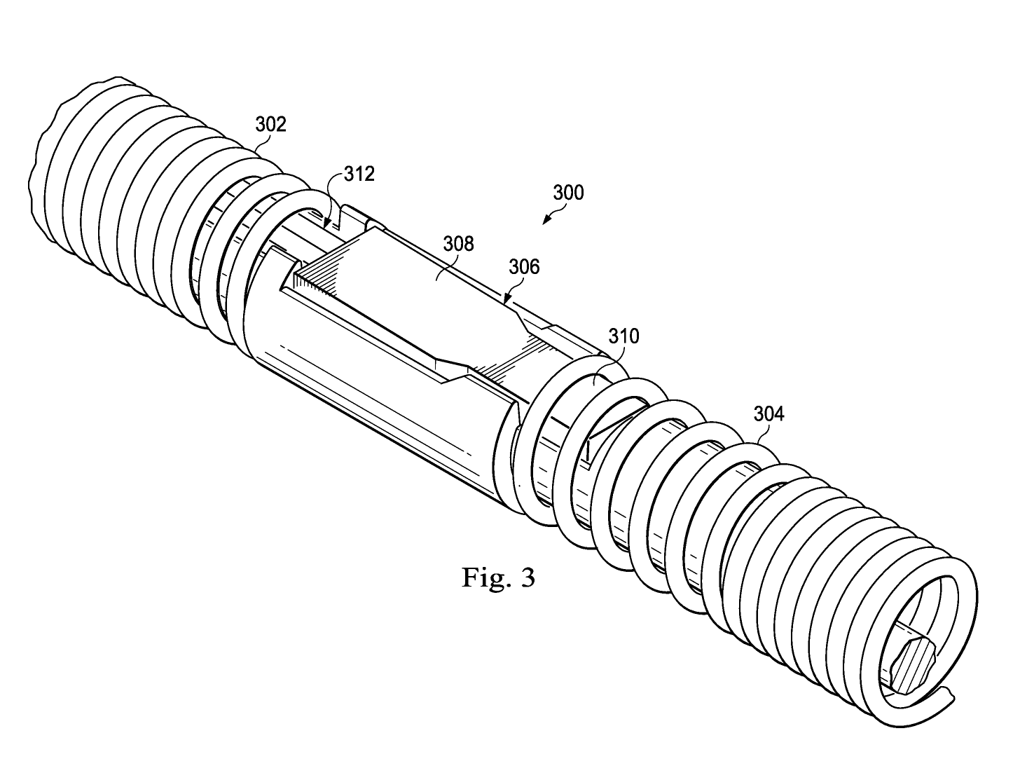

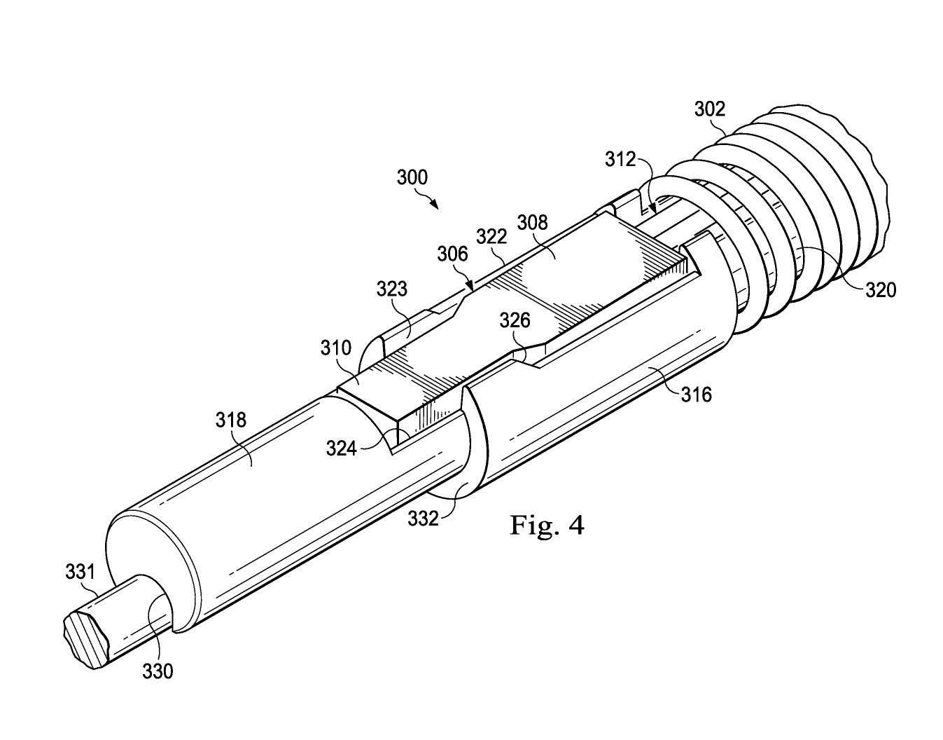

Referring now to FIG. 2, shown therein is a cross-sectional side view of an intravascular device 200 according to an embodiment of the present disclosure. In that regard, the intravascular device 200 is provided as an exemplary embodiment of the type of intravascular device into which the mounting structures, including the associated structural components and methods, described below with respect to FIGS. 3-12 can be implemented. However, it is understood that no limitation is intended thereby and that the concepts of the present disclosure are applicable to a wide variety of intravascular devices, including those described in U.S. Pat. No. 7,967,762 and U.S. Patent Application Publication No. 2009/0088650, each of which is hereby incorporated by reference in its entirety.

As shown in FIG. 2, the intravascular device 200 includes a proximal portion 202, a middle portion 204, and a distal portion 206. Generally, the proximal portion 202 is configured to be positioned outside of a patient, while the distal portion 206 and a majority of the middle portion 204 are configured to be inserted into the patient, including within human vasculature. In that regard, the middle portion 204 and/or distal portion 206 have an outer diameter between about 0.0007″ (0.0178 mm) and about 0.118″ (3.0 mm) in some embodiments, with some particular embodiments having an outer diameter of approximately 0.014″ (0.3556 mm) or approximately 0.018″ (0.4572 mm)). In the illustrated embodiment of FIG. 2, the middle and distal portions 204, 206 of the intravascular device 200 each have an outer diameter of 0.014″ (0.3556 mm).

As shown, the distal portion 206 of the intravascular device 200 has a distal tip 207 defined by an element 208. In the illustrated embodiment, the distal tip 207 has a rounded profile. In some instances, the element 208 is radiopaque such that the distal tip 207 is identifiable under x-ray, fluoroscopy, and/or other imaging modalities when positioned within a patient. In some particular instances, the element 208 is solder secured to a flexible element 210 and/or a flattened tip core 212. In that regard, in some instances the flexible element 210 is a coil spring. The flattened tip core 212 extends distally from a distal portion of a core 214. As shown, the distal core 214 tapers to a narrow profile as it extends distally towards the distal tip 207. In some instances, the distal core 214 is formed of a stainless steel that has been ground down to have the desired tapered profile. In some particular instances, the distal core 214 is formed of high tensile strength 304V stainless steel. In an alternative embodiment, the distal core 214 is formed by wrapping a stainless steel shaping ribbon around a nitinol core. In some embodiments, the distal core 214 is secured to a mounting structure 218 by mechanical interface, solder, adhesive, combinations thereof, and/or other suitable techniques as indicted by reference numerals 216. The mounting structure 218 is configured to receive and securely hold a component 220. In that regard, the component 220 is one or more of an electronic component, an optical component, and/or electro-optical component. For example, without limitation, the component 220 may be one or more of the following types of components: a pressure sensor, a temperature sensor, an imaging element, an optical fiber, an ultrasound transducer, a reflector, a minor, a prism, an ablation element, an RF electrode, a conductor, and/or combinations thereof.

The mounting structure 218 is fixedly secured within the distal portion 206 of the intravascular device 200. As will be discussed below in the context of the exemplary embodiments of FIGS. 3-12, the mounting structure 218 may be fixedly secured to a core wire (i.e., a single core running along the length of the mounting structure), flexible elements or other components surrounding at least a portion of the mounting structure (e.g., coils, polymer tubing, etc.), and/or other structure(s) of the intravascular device positioned adjacent to the mounting structure. In the illustrated embodiment, the mounting structure is disposed at least partially within flexible element 210 and/or a flexible element 224 and secured in place by an adhesive or solder 222. In some embodiments, the mounting structure 218 is disposed entirely within flexible element 210 and/or flexible element 224. In some instances, the flexible elements 210 and 224 are flexible coils. In one particular embodiment, the flexible element 224 is ribbon coil covered with a polymer coating. For example, in one embodiment the flexible element 224 is a stainless steel ribbon wire coil coated with polyethylene terephthalate (PET). In another embodiment, the flexible element is a polyimide tubing that has a ribbon wire coil embedded therein. An adhesive is utilized to secure the mounting structure 218 to the flexible element 210 and/or the flexible element 224 in some implementations. Accordingly, in some instances the adhesive is urethane acrylate, cyanoacrylate, silicone, epoxy, and/or combinations thereof.

The mounting structure 218 is also secured to a core 226 that extends proximally from the mounting structure towards the middle portion 204 of the intravascular device 200. In that regard, core 226 and distal core 214 are integrally formed in some embodiments such that a continuous core passes through the mounting structure. In the illustrated embodiment, a portion 228 of the core 226 tapers as it extends distally towards mounting structure 218. However, in other embodiments the core 226 has a substantially constant profile along its length. In some implementations, the diameter or outer profile (for non-circular cross-sectional profiles) of core 226 and core 214 are the same Like distal core 214, the core 226 is fixedly secured to the mounting structure 218. In some instances, solder and/or adhesive is used to secure the core 226 to the mounting structure 218. In the illustrated embodiment, solder/adhesive 230 surrounds at least a part of the portion 228 of the core 226. In some instances, the solder/adhesive 230 is the solder/adhesive 222 used to secure the mounting structure 218 to the flexible element 210 and/or flexible element 224. In other instances, solder/adhesive 230 is a different type of solder or adhesive than solder/adhesive 222. In one particular embodiment, adhesive or solder 222 is particularly suited to secure the mounting structure 218 to flexible element 210, while solder/adhesive 230 is particularly suited to secure the mounting structure to flexible element 224.

A communication cable 232 extends along the length of the intravascular device 200 from the proximal portion 202 to the distal portion 206. In that regard, the distal end of the communication cable 232 is coupled to the component 220 at junction 234. The type of communication cable utilized is dependent on the type of electronic, optical, and/or electro-optical components that make up the component 220. In that regard, the communication cable 232 may include one or more of an electrical conductor, an optical fiber, and/or combinations thereof. Appropriate connections are utilized at the junction 234 based on the type of communication lines included within communication cable 232. For example, electrical connections are soldered in some instances, while optical connections pass through an optical connector in some instances. In some embodiments, the communication cable 232 is a trifilar structure, a bifilar structure, a single conductor (which may be a conductive core or a conductor separate from the core). Further, it is understood that all and/or portions of each of the proximal, middle, and/or distal portions 202, 204, 206 of the intravascular device 200 may have cross-sectional profiles as shown in FIGS. 2-5 of U.S. Provisional Patent Application No. 61/665,697 filed on Jun. 28, 2012, which is hereby incorporated by reference in its entirety.

Further, in some embodiments, the proximal portion 202 and/or the distal portion 206 incorporate spiral ribbon tubing as disclosed in U.S. Provisional Patent Application No. 61/665,697 filed on Jun. 28, 2012. In some instances, the use of such spiral ribbon tubing allows a further increase in the available lumen space within the device. For example, in some instances use of a spiral ribbon tubing having a wall thickness between about 0.001″ and about 0.002″ facilitates the use of a core wire having an outer diameter of at least 0.0095″ within a 0.014″ outer diameter guide wire using a trifilar with circular cross-sectional conductor profiles. The size of the core wire can be further increased to at least 0.010″ by using a trifilar with the flattened oblong cross-section conductor profiles. The availability to use a core wire having an increased diameter allows the use of materials having a lower modulus of elasticity than a standard stainless steel core wire (e.g., superelastic materials such as Nitinol or NiTiCo are utilized in some instances) without adversely affecting the handling performance or structural integrity of the guide wire and, in many instances, provides improvement to the handling performance of the guide wire, especially when a superelastic material with an increased core diameter (e.g., a core diameter of 0.0075″ or greater) is utilized within the distal portion 206.

The distal portion 206 of the intravascular device 200 also optionally includes at least one imaging marker 236. In that regard, the imaging marker 236 is configured to be identifiable using an external imaging modality, such as x-ray, fluoroscopy, angiograph, CT scan, MRI, or otherwise, when the distal portion 206 of the intravascular device 200 is positioned within a patient. In the illustrated embodiment, the imaging marker 236 is a radiopaque coil positioned around the tapered distal portion 228 of the core 226. Visualization of the imaging marker 236 during a procedure can give the medical personnel an indication of the size of a lesion or region of interest within the patient. To that end, the imaging marker 236 can have a known length (e.g., 0.5 cm or 1.0 cm) and/or be spaced from the element 218 by a known distance (e.g., 3.0 cm) such that visualization of the imaging marker 236 and/or the element 218 along with the anatomical structure allows a user to estimate the size or length of a region of interest of the anatomical structure. It is understood that a plurality of imaging markers 236 are utilized in some instances. In that regard, in some instances the imaging markers 236 are spaced a known distance from one another to further facilitate measuring the size or length of the region of interest.

In some instances, a proximal portion of the core 226 is secured to a core 238 that extends through the middle portion 204 of the intravascular device. In that regard, the transition between the core 226 and the core 238 may occur within the distal portion 206, within the middle portion 204, and/or at the transition between the distal portion 206 and the middle portion 204. For example, in the illustrated embodiment the transition between core 226 and core 238 occurs in the vicinity of a transition between the flexible element 224 and a flexible element 240. The flexible element 240 in the illustrated embodiment is a hypotube. In some particular instances, the flexible element is a stainless steel hypotube. Further, in the illustrated embodiment a portion of the flexible element 240 is covered with a coating 242. In that regard, the coating 242 is a hydrophobic coating in some instances. In some embodiments, the coating 242 is a polytetrafluoroethylene (PTFE) coating.

The proximal portion of core 226 is fixedly secured to the distal portion of core 238. In that regard, any suitable technique for securing the cores 226, 238 to one another may be used. In some embodiments, at least one of the cores 226, 238 includes a plunge grind or other structural modification that is utilized to couple the cores together. In some instances, the cores 226, 238 are soldered together. In some instances, an adhesive is utilized to secure the cores 226, 238 together. In some embodiments, combinations of structural interfaces, soldering, and/or adhesives are utilized to secure the cores 226, 238 together. In other instances, the core 226 is not fixedly secured to core 238. For example, in some instances, the core 226 and the core 246 are fixedly secured to the hypotube 240 and the core 238 is positioned between the cores 226 and 246, which maintains the position of the core 238 between cores 226 and 246. In some implementations, the cores 226, 238, and 246 are integrally formed as a single core.

In some embodiments, the core 238 is formed of a different material than the core 226. For example, in some instances the core 226 is formed of nitinol and the core 238 is formed of stainless steel. In other instances, the core 238 and the core 226 are formed of the same material. In some instances the core 238 has a different profile than the core 226, such as a larger or smaller diameter and/or a non-circular cross-sectional profile. For example, in some instances the core 238 has a D-shaped cross-sectional profile. In that regard, a D-shaped cross-sectional profile has some advantages in the context of an intravascular device 200 that includes one or more electronic, optical, or electro-optical component in that it provides a natural space to run any necessary communication cables while providing increased strength than a full diameter core. In other instances, core 238 and core 226 are made of the same material and/or have the same structure profiles such that the cores 226 and 238 form a continuous, monolithic core.

In some instances, a proximal portion of the core 238 is secured to a core 246 that extends through at least a portion of the proximal portion 202 of the intravascular device 200. In that regard, the transition between the core 238 and the core 246 may occur within the proximal portion 202, within the middle portion 204, and/or at the transition between the proximal portion 202 and the middle portion 204. For example, in the illustrated embodiment the transition between core 238 and core 246 is positioned distal of a plurality of conducting bands 248. In that regard, in some instances the conductive bands 248 are portions of a hypotube. Proximal portions of the communication cable 232 are coupled to the conductive bands 248. In that regard, in some instances each of the conductive bands is associated with a corresponding communication line of the communication cable 232. For example, in embodiments where the communication cable 232 consists of a trifilar, each of the three conductive bands 248 are connected to one of the conductors of the trifilar, for example by soldering each of the conductive bands to the respective conductor. Where the communication cable 232 includes optical communication line(s), the proximal portion 202 of the intravascular device 200 includes an optical connector in addition to or instead of one or more of the conductive bands 248. An insulating layer or sleeve 250 separates the conductive bands 248 from the core 246. In some instances, the insulating layer 250 is formed of polyimide.

As noted above, the proximal portion of core 238 is fixedly secured to the distal portion of core 246. In that regard, any suitable technique for securing the cores 238, 246 to one another may be used. In some embodiments, at least one of the cores includes a structural feature that is utilized to couple the cores together. In the illustrated embodiment, the core 238 includes an extension 252 that extends around a distal portion of the core 246. In some instances, the cores 238, 246 are soldered together. In some instances, an adhesive is utilized to secure the cores 238, 246 together. In some embodiments, combinations of structural interfaces, soldering, and/or adhesives are utilized to secure the cores 238, 246 together. In other instances, the core 226 is not fixedly secured to core 238. For example, in some instances and as noted above, the core 226 and the core 246 are fixedly secured to the hypotube 240 and the core 238 is positioned between the cores 226 and 246, which maintains the position of the core 238 between cores 226 and 246. In some embodiments, the core 246 is formed of a different material than the core 238. For example, in some instances the core 246 is formed of Nitinol and/or NiTiCo (nickel-titanium-cobalt alloy) and the core 238 is formed of stainless steel. In that regard, by utilizing a nitinol core within the conductive bands 248 instead of a stainless steel the likelihood of kinking is greatly reduced because of the increased flexibility of the nitinol core compared to a stainless steel core. In other instances, the core 238 and the core 246 are formed of the same material. In some instances the core 238 has a different profile than the core 246, such as a larger or smaller diameter and/or a non-circular cross-sectional profile. In other instances, core 238 and core 246 are made of the same material and/or have the same structure profiles such that the cores 238 and 246 form a continuous, monolithic core.

Referring now to FIGS. 3-12, shown therein are aspects of various embodiments of mounting structures for use within intravascular devices and associated methods. In some embodiments, the mounting structures of the present disclosure are sized and shaped for use within guide wires having a diameter of 0.018″ or 0.014″. Referring initially to FIGS. 3-7, shown therein is a mounting structure 300. As will be discussed below, mounting structure 300 is configured for use with a core that extends along the length of the mounting structure. Accordingly, in some embodiments the mounting structure 300 is utilized as mounting structure 218 of intravascular device 200 discussed above, where distal core 214 and proximal core 226 are defined by a single core that extends along and/or through mounting structure 300. However, in some implementations separate proximal and distal cores are utilized as discussed above with respect to distal core 214 and proximal core 226. In some implementations, at least the portion of the core running along the length of the mounting structure 300 has a constant profile. In other implementations, at least the portion of the core running along the length of the mounting structure 300 has a variable profile (e.g., tapered or stepped along its length). Accordingly, it is understood that the recesses and openings discussed below that receive the core may likewise have constant and/or variable profiles along their length.

As shown in FIG. 3, in some embodiments the mounting structure 300 is implemented within a distal portion of a guide wire having a proximal coil 302 and a distal coil 304. In that regard, a proximal portion of the mounting structure 300 is positioned within and serves as an alignment feature for the proximal coil 302, while a distal portion of the mounting structure 300 is positioned within and serves as an alignment feature for the distal coil 304. In some other implementations, the mounting structure is positioned within a single coil. In that regard, in some implementations the coil pitch is varied along the length of the coil to provide access to access to a sensing component 306 discussed in more detail below. Further, when the mounting structure is positioned within a single coil the mounting structure may include a generally constant outer profile (e.g., maximum outer diameter) along its length (i.e., does not include the reduced diameter portions for interfacing with the proximal and distal coils 302, 304 as shown in the illustrated embodiment). Further still, it should be noted that in some instances the proximal coil 302 and distal coil 304 interface with one another (or come into close proximity to one another), such that the mounting structure 300 is fully received within the proximal and distal coils. In such instances, the mounting structure may again have a generally constant outer profile (e.g., maximum outer diameter) along its length.

A sensing component 306 is mounted to the mounting structure 300. In the illustrated embodiment of FIG. 3, the sensing component 306 is a pressure sensor mounted in a face down configuration. In that regard, the sensing component 306 includes a main body 308 and a cantilevered portion 310 extending from the main body 308. In some implementations, a diaphragm of the pressure sensor is formed on the cantilevered portion 310. Thus, when the pressure sensor is mounted in the face down configuration of FIG. 3, the diaphragm faces towards an inner portion of the mounting structure 300. Accordingly, in some embodiments an opening extends through the mounting structure 300 from a surface adjacent to the cantilevered portion 310 (e.g., top of the mounting structure as viewed in FIG. 3) to an opposing surface opposite the cantilevered portion 310 (e.g., bottom of the mounting structure as viewed in FIG. 3). Such an opening is utilized to expose the diaphragm of the pressure sensor to ambient in some implementations. In some instances, the opening extends perpendicular to a longitudinal axis of the mounting structure. As shown in FIG. 3, in the illustrated embodiment at least a section of the cantilevered portion 310 is covered by a proximal section of coil 304. In that regard, the coil 304 provides physical protection to the sensing component 306. Further, the spacing between the windings of the coil 304 ensures that the pressure sensing components are exposed to ambient pressure.

In some instances, the sensing component 306 is mounted such that there is space between sidewalls of the mounting structure 300 and the cantilevered portion 310. Such spacing can both expose the diaphragm to ambient as well as promote the escape of any air bubbles that may become trapped on the diaphragm surface. In that regard, the spacing between the sidewalls of the mounting structure 300 and the cantilevered portion 310 may be accomplished through vertical spacing (i.e., the bottom of the cantilevered portion 310 is higher than the top of one or both of the adjacent sidewalls of the mounting structure), lateral spacing (i.e., the width of the cantilevered portion 310 is less than a width between the opposing sidewalls adjacent the cantilevered portion such that a space is created between one or both sides of the cantilevered portion and the adjacent sidewall(s)), and/or combinations thereof (i.e., both vertical and lateral spacings). Spacing the cantilevered portion 310 from the sidewalls of the mounting structure 300 is particularly suitable for implementations of face-down mounting of a sensing element.

The sensing component 306 is coupled to communication lines 312. In the illustrated embodiment, which implements a pressure sensor as the sensing component, communication lines 312 consist of three electrical leads (commonly referred to as a trifilar). However, the type of communication line utilized is dependent on the type of electronic, optical, and/or electro-optical elements that make up the sensing component 306. In that regard, the communication lines 312 may include one or more of an electrical conductor, an optical fiber, and/or combinations thereof. Appropriate connections are utilized to secure the communications lines 312 to the sensing component 306 based on the type of communication lines utilized. For example, electrical connections are soldered in some instances, while optical connections pass through an optical connector in some instances.

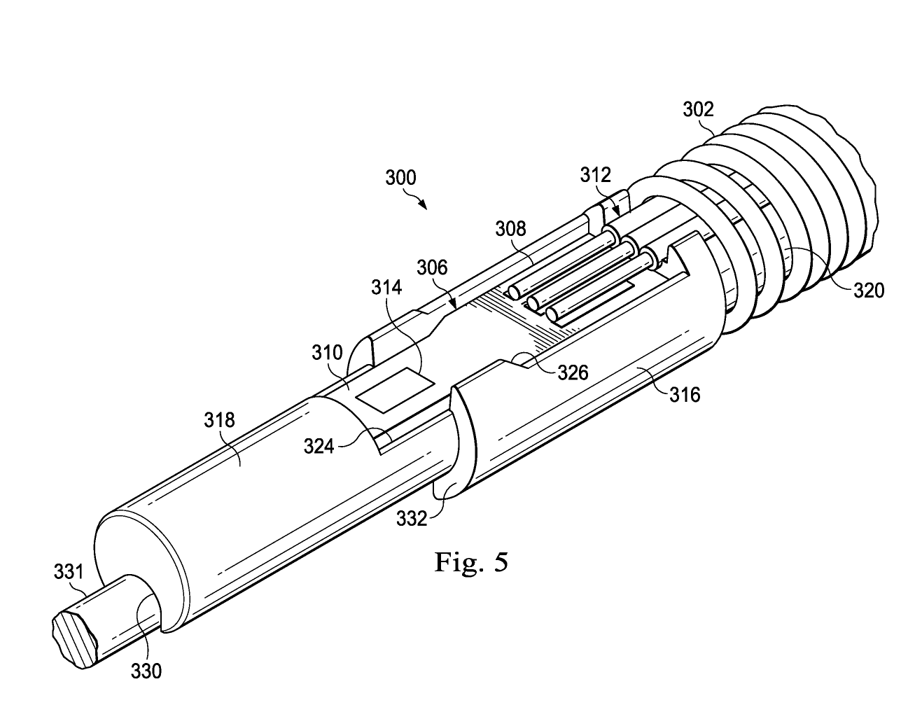

While FIGS. 3 and 4 show the sensing component 306 mounted in a face down configuration, the mounting structure 300 also facilitates mounting the sensing component 306 in a face up configuration as shown in FIG. 5. In the illustrated embodiment, the sensing component 306 is a pressure sensor having a diaphragm 314. Accordingly, when the pressure sensor is mounted in the face up configuration of FIG. 5, the diaphragm 314 faces outward, away from the mounting structure 300. In some implementations, at least a section of the cantilevered portion 310 that includes the diaphragm 314 is covered by a proximal section of coil 304. In that regard, the coil 304 provides physical protection to the sensing component 306, while the spacing between the windings of the coil 304 ensures that the diaphragm 314 is exposed to ambient pressure.

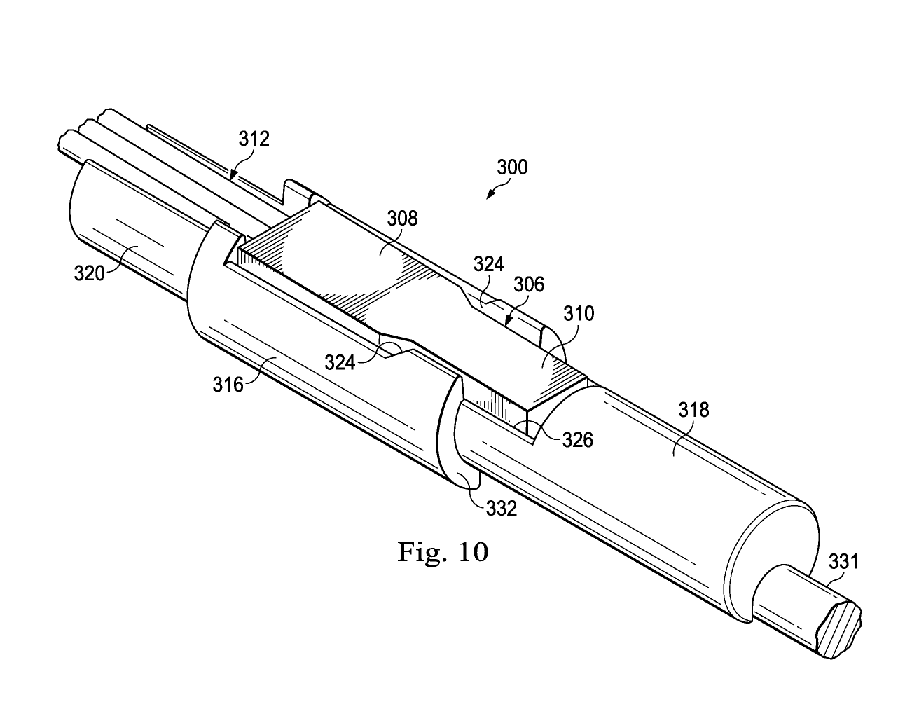

As shown in FIGS. 4-7 and 9, the mounting structure 300 has various structural features to facilitate interfacing with other components of the intravascular device. In the illustrated embodiment, the mounting structure 300 includes a central portion 316, a distal portion 318, and a proximal portion 320. In that regard, the distal portion 318 is configured to interface with coil 304, while proximal portion 320 is configured to interface with coil 302. Generally, the central portion 316 has a diameter that is equal to or less than the outer diameter of the guide wire and equal to or larger than the diameters of distal portion 318 and proximal portion 320. In some implementations, the central portion 316 has a length 1 mm or less. Further, the central portion 316 and the distal portion 318 collectively define a mounting area for the sensing component 306, while the proximal portion 320 provides an area for the communication lines 312 to extend proximally from the mounted sensing component. In some implementations, the central and distal portions 316, 318 define a recess or opening configured to receive the sensing component 306 of the intravascular device and/or communication lines coupled to the sensing component. In the illustrated embodiment, the recess is particularly suited for use with a pressure sensing element and a trifilar communication cable. As shown in FIGS. 4 and 9, the recess includes a widened portion defined by sidewalls 322 of the central portion 316 and a narrowed portion defined by sidewalls 323 and 324 of the central and distal portions 316, 318, respectively. Accordingly, in some implementations the portion defined by sidewalls 322 is sized and shaped to receive a main body of a pressure sensing element, while the portion defined by sidewalls 323 and 324 is sized and shaped to receive a portion of an active portion of the pressure sensing element (e.g., a cantilevered structure including a pressure-sensing diaphragm). To that end, in some implementations the sidewalls 322 may contact the main body 308 of the sensing component 306 when the sensing component is seated within the recess, but the cantilevered portion 310 is always spaced from the sidewalls 323 and 324 by design of the recess or opening profile.

In that regard, as shown in FIG. 9, in some instances the recess includes a surface 327 for the main body of the pressure sensing element to be mounted to. Further, within surface 327 is an opening 328. In that regard, in some implementations of face down mounting of the sensing component 306, opening 328 provides space for the connection of the communication lines 312 to the sensing component and coupling of a core to the mounting structure 300. For example, where communication lines 312 are soldered and/or covered with an encapsulant at the connection to the sensing component 306, an increased thickness often results. Opening 328 provides a space for the increased material thickness to be disposed so that a planar surface of the sensing component 306 can be seated flat or co-planar along surface 327. In some instances, opening 328 defines a mold cavity that is at least partially, including fully, filled with an epoxy or adhesive, such as Ablebond, that secures the core, mounting structure, and sensing component to one another. In some embodiments, a non-conductive moisture inhibiting encapsulant seals the connections between the communication lines 312 and the sensing component 306 from environmental exposure during use and also bonds together the sensing component 306, communication lines 312, mounting structure 330, and core 331.

Further, an opening 329 creates a space within mounting structure 300 that can be utilized to expose a diaphragm of a pressure sensor that is mounted in a face down configuration to ambient. In that regard, the opening 329 extends all of the way through the mounting structure 300 in some instances. In other instances, the opening 329 extends only partially through the mounting structure 300 and the diaphragm is exposed to ambient as a result of spacing, either vertically or horizontally, the sensing component 306 from the sidewalls of the mounting structure. In some instances, the opening 329 extends all of the way through the mounting structure and the sensing component is spaced from the sidewalls.

A transition or taper 326 extends between the sidewalls 322 and the sidewalls 323. In that regard, the transition or taper 326 is utilized in some embodiments to properly align and seat the sensing component 306 within the mounting structure. In that regard, it is understood that the mounting structure 300 and the sensing component 306 have mating and/or complimentary features to facilitate alignment in some embodiments. For example, one or both of the mounting structure 300 and the sensing component 306 may have projections, recesses, openings, detents, tapers, other structural features, and/or combinations thereof that are utilized to properly align the sensing component 306 with respect to the mounting structure. Further, in some embodiments the mounting structure 300 includes one or more angled or tapered inner walls that are suitable for guiding the sensing component 306 into a desired mounting position within the mounting structure. In that regard, the angled or tapered inner walls facilitate easier assembly in some instances by allowing the initial placement of the sensing component 306 to be less precise, but still resulting in a very precise placement of the sensing component due to the angled or tapered surfaces guiding the sensing component 306 to the desired mounting location. In some instances, the mating or complimentary features of the mounting structure 300 and the sensing component 306 serve as a stop to the guided placement of the angled or tapered inner walls. In other words, the mating or complimentary features of the mounting structure 300 and the sensing component 306 will interface when the sensing component has reached the desired mounting position. The structural design of the mounting structure 300 is generally designed to ensure that an active portion of the sensing component (e.g., a portion containing the diaphragm or other pressure sensing structure) is spaced from all surfaces of the mounting structure when the sensing component is seated into the mounting structure.

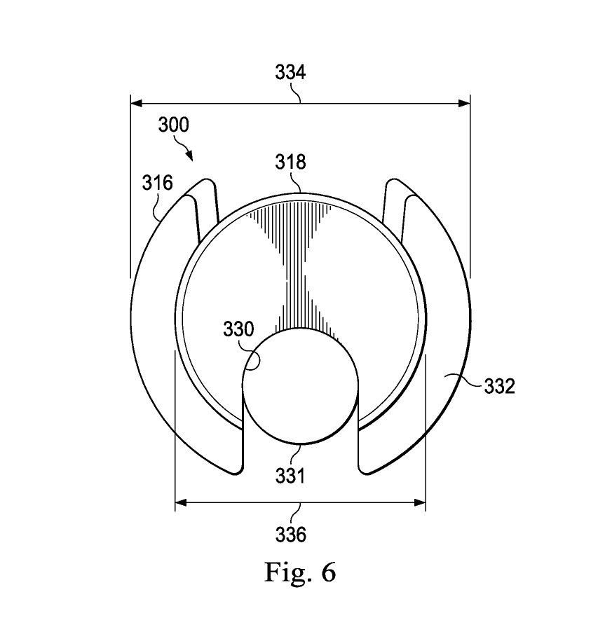

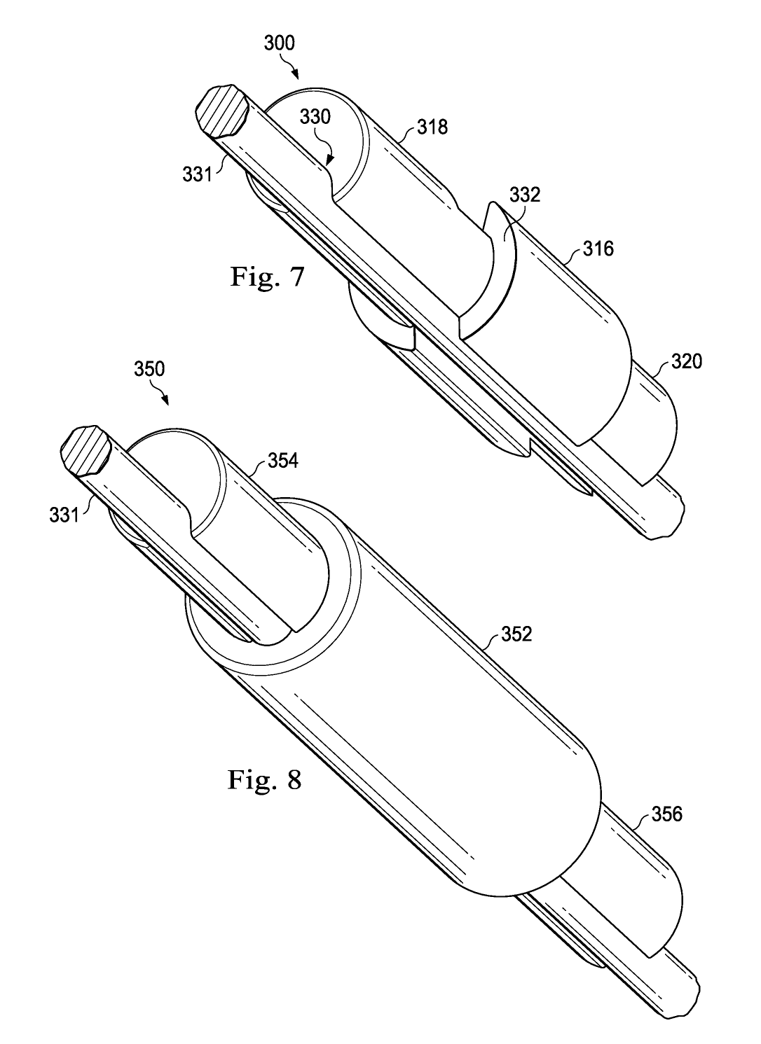

As best seen in FIGS. 6 and 7, the mounting structure 300 also includes a recess or opening 330 that extends along the length of the mounting structure 300 between the distal portion 318 and the proximal portion 320. In that regard, the recess or opening 330 is sized and shaped to interface with a core wire. Accordingly, in some instances the recess/opening 330 has an outer diameter or width (e.g., for non-circular cross-sectional profiles) between about 0.09 mm and about 0.12 mm, with some particular embodiments tapering from 0.115 mm (proximal diameter) to 0.111 mm (distal diameter). In some instances, the core wire is positioned within the recess/opening 330 and then fixedly secured into place using solder, adhesive, and/or other suitable techniques. In that regard, in some instances the core 331 is positioned within the recess/opening 330 by being advanced axially along and through the recess/opening 330. In other instances, the core 331 is positioned within the recess/opening 330 by being advanced in a direction perpendicular to the longitudinal axis of the mounting structure and the recess/opening 330. Further, the recess/opening 330 may be positioned such that when the core is positioned within the recess/opening 330, the core is coaxial with a central longitudinal axis of the mounting structure 300 or the core is radially offset with respect to the central longitudinal axis of the mounting structure (as shown in FIG. 6). In that regard, having the core offset creates a natural space within the mounting structure 300 for placement of the sensing component 306, which also prevents the need to create a custom profile for the core to facilitate placement of the sensing component in a desired manner (e.g., cantilevering a pressure sensor).

In some instances, the recess/opening 330 has a constant outer profile (e.g., diameter) along its length. In other instances, the recess/opening 330 has a variable outer profile along its length. For example, in some embodiments the recess/opening 330 is tapered along its length (e.g., from a larger diameter to a smaller diameter as it extends distally from proximal portion 320 to distal portion 318). In other embodiments, the recess/opening 330 has a variable outer profile that is stepped along its length. In some instances, the outer profile of the recess/opening 330 is tapered, stepped, or otherwise varied to match a corresponding change in the outer profile of the core that will be positioned within the recess/opening. For example, in one particular embodiment the with some particular embodiments a diameter of the recess/opening tapers from 0.115 mm to about 0.111 mm as the recess/opening extending proximally to distally along the axial length of the recess/opening.

Further, while a mounting structure has generally been described as a component that is (micro)molded, machined, printed, and/or otherwise formed as a discrete component then attached to the core wire. The mounting structure can also be molded, machined, printed, and/or otherwise formed directly onto the core wire. For example, this is performed in some instances by fixing the bare core wire into a (micro)mold cavity and forming the structure directly onto the core wire. In another embodiment, the core wire is formed as two separate structures with the mounting structure serving as a bridge between a proximal core portion and a distal core portion. In such an embodiment, the mounting structure can be a discrete component separate from both the proximal and distal core portions, formed/over-molded onto the proximal core portion, then secured to the distal core portion, formed/over-molded onto the distal core portion, then secured to the proximal core portion, or formed/over-molded onto both the proximal and distal core portions. Similarly, the mounting structure itself consists of two elements in some instances. For example, in some implementations the mounting structure includes a pedestal portion that is attached to the core wire(s) as described above and a sensor portion with an over-molded cap that, when mated to the pedestal portion, forms a mounting structure having a generally uniform outer diameter. In that regard, when mated a proximal section of the sensor portion is secured between the pedestal portion and the over-molded cap, while a distal section of the sensor portion is spaced from at least the pedestal portion.

As shown in FIG. 6, with the core 331 mounted within the recess/opening 330, a section of the outer surface of the core 331 (i.e., bottom section of the core 331 in FIG. 6) is generally aligned with a circumference defined by the outer surface of distal portion 318. In this manner, the section of the outer surface of the core 331 can be considered to complete or fill in the gap in the circumference or outer profile of the distal portion 318 that is created by recess/opening 330. Accordingly, with the core 331 mounted within the recess/opening 330, the core 331 and distal portion 318 define an alignment feature for mounting the distal coil 304 (as shown in FIG. 3) to the mounting structure 300. In that regard, the outer circumference defined by the core 331 and distal portion 318 is sized and shaped to be received within the inner circumference of the coil 304. In some instances, a surface 332 extending perpendicular to the longitudinal axis of the mounting structure 300 serves as a stop for the coil 304. In that regard, the coil 304 is advanced along the distal portion 318 of the mounting structure 300 until it contacts the surface 332 in some instances. In the illustrated embodiment, surface 332 is defined by the transition between central portion 316 and distal portion 318. With the coil 304 properly aligned and positioned over the distal portion 318 and core 331, the coil 304 is secured to the mounting structure 300 and/or core 331. In some implementations the coil 304 is secured using solder, adhesive, and/or combinations thereof. In some implementations, at least a portion of an outer surface of the distal portion 318 includes threaded recesses sized and shaped to allow a portion of the coil 304 to be threaded onto the distal portion 318 of the mounting structure.

Similarly, with the core 331 mounted within the recess/opening 330, the core 331 and proximal portion 320 define an alignment feature for mounting the proximal coil 302 (as shown in FIG. 3) to the mounting structure 300. In that regard, the outer circumference defined by the core 331 and distal portion 318 is sized and shaped to be received within the inner circumference of the coil 302. In some instances, a surface extending perpendicular to the longitudinal axis of the mounting structure 300, similar to surface 332 described above, serves as a stop for the coil 302. In that regard, the coil 302 is advanced along the proximal portion 320 of the mounting structure 300 until it contacts the surface in some instances. In some instances, the stopping surface is defined by the transition between central portion 316 and proximal portion 320. Coils 302 and 304 may have the same or different inner circumferences. Accordingly, the distal and proximal portions 318, 320 may have the same or different outer profiles. With the coil 302 properly aligned and positioned over the proximal portion 320 and core 331, the coil 302 is secured to the mounting structure 300 and/or core 331. In some implementations the coil 302 is secured using solder, adhesive, and/or combinations thereof. In some implementations, at least a portion of an outer surface of the proximal portion 320 includes threaded recesses sized and shaped to allow a portion of the coil 302 to be threaded onto the proximal portion 320 of the mounting structure. In other embodiments, in addition to or in lieu of the threaded recesses, the proximal and/or distal portions of the mounting structure 300 include other structure features for engaging with the proximal coil and/or distal coil, such as bumps, ribs, roughened surfaces, sawteeth, and/or other suitable engagement features.

As shown in FIGS. 4-7 and 9, the central portion 316 has a larger outer profile than the distal and proximal portions 318, 320. In the illustrated embodiment, each of the central portion 316, distal portion 318, and proximal portion 320 have generally circular cross-sectional profiles such that the outer profiles are defined by a diameter. In that regard, in some instances, the diameter 334 of the central portion 316, as shown in FIG. 6, is between about 0.25 mm and about 0.35 mm, with some particular embodiments having a diameter of 0.25 mm and 0.29 mm. Further, the diameter 336 of the distal portion 318, also shown in FIG. 6, is between about 0.25 mm and about 0.35 mm, with some particular embodiments having a diameter of 0.25 mm and 0.29 mm. Further still, the diameter of the distal portion 320 is between about 0.25 mm and about 0.35 mm, with some particular embodiments having a diameter of 0.25 mm and 0.29 mm. In that regard, in some implementations the distal and proximal portions 318, 320 have the same diameter or outer profile. In other implementations, the distal and proximal portions 318, 320 have different diameters and/or outer profiles. It is understood that in some embodiments one or more of the central, distal, and proximal portions 316, 318, and 320 have a non-circular cross-sectional profile, including geometric and non-geometric cross-sectional profiles. In some such embodiments, the sides of the mounting structure 300 have an overall rounded or arcuate profile, while at least one of the upper and lower surfaces of the mounting structure is flattened or planar. In that regard, the radius or rate of curvature of the rounded/arcuate sides is determined based on the desired outer diameter (e.g., 0.014″, 0.018″, etc.) of the guide wire into which the mounting structure 300 will be incorporated. As shown in FIG. 9, the mounting structure 300 also has a length 338 between its proximal and distal ends. In some embodiments, the length 338 is between about 0.50 mm and about 2.00 mm, with some particular embodiments having a length of 0.50 mm and 1.80 mm. In some instances, the central portion 316 has a length between about 0.01 mm and about 1.0 mm.

Generally, the mounting structure 300 can be made of any suitable biocompatible material. For example, the mounting structures of the present disclosure may be formed from a conductive material (e.g., Stainless Steel (17-4, 316, 430, 304), Soft Magnetic Alloys (Fe-50% Co, Fe-3% Si, 4-79 Moly Permalloy®, Fe-50% Ni), Controlled Expansion Alloys (ASTM F-15 [Fe—Ni—Co], Fe-42% Ni), Low Alloy Steel (7% Ni-Fe), Tungsten Heavy Alloy, Titanium, and/or other suitable conductive material), a non-conductive material (e.g., HDPE, PP, POM, LCP, and/or other suitable non-conductive material), a rigid material (e.g., Stainless Steel (17-4, 316, 430, 304), Soft Magnetic Alloys (Fe-50% Co, Fe-3% Si, 4-79 Moly Permalloy®, Fe-50% Ni), Controlled Expansion Alloys (ASTM F-15 [Fe—Ni—Co], Fe-42% Ni), Low Alloy Steel (7% Ni—Fe), Tungsten Heavy Alloy, Titanium, HDPE, PP, POM, LCP, and/or other suitable rigid material), a pliable material (e.g., silicone and/or other suitable pliable material), and/or combinations thereof. Accordingly, the mounting structures of the present disclosure may be manufactured using any suitable technique, including without limitation micro-machining, micro-EDM, micro-laser, micro-molding, stamping, LIGA, and/or combinations thereof.

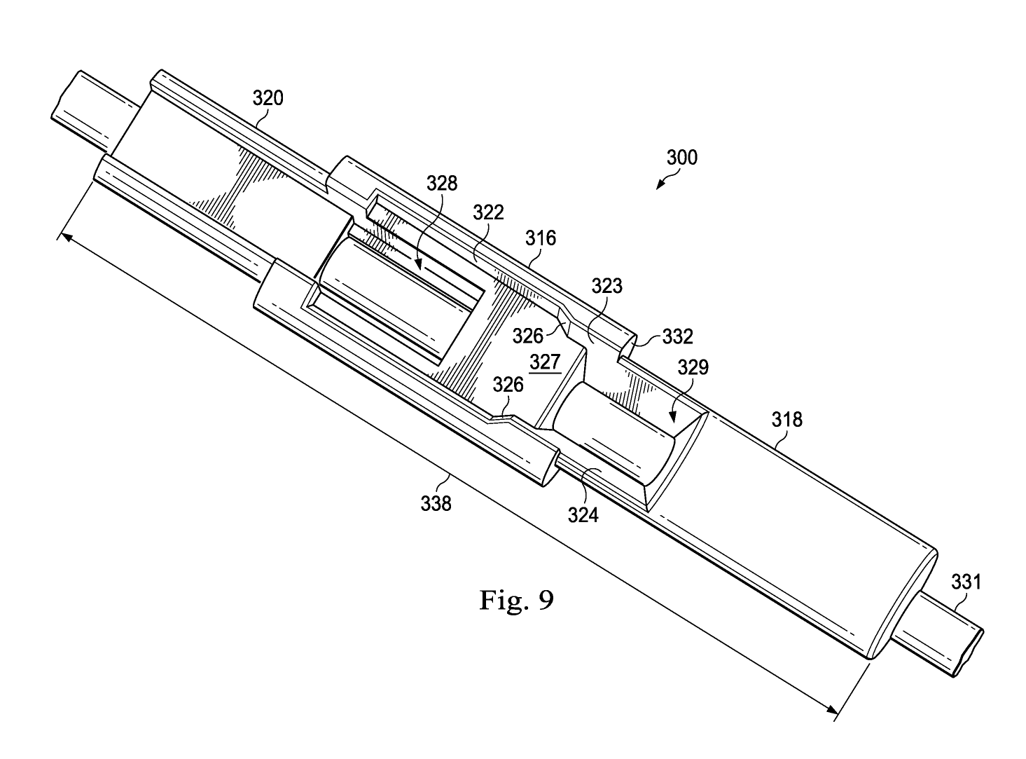

FIG. 8 illustrates an embodiment of a mounting structure 350 according to another embodiment of the present disclosure. In that regard, mounting structure 350 is similar to mounting structure 300 in most respects except that mounting structure 350 completely surrounds at least a portion of the core 331. As shown, the mounting structure 350 includes a central portion 352, a distal portion 354, and a proximal portion 356. In that regard, the central portion 352 completely surrounds the core 331, while the distal portion 354 and proximal portion 356 partially surround the core 331. In some instances, the distal and proximal portions 354, 356 partially surround the core 331 such that a section of an outer surface of the core completes the circumference or outer boundary of the distal and proximal portions. In some embodiments the core 331 is positioned within a mold and the mounting structure 350 is injection molded around the core 331. In other embodiments, the mounting structure 350 is formed separately—with an opening extending through the central portion 352 that is in communication and alignment with recesses/openings in the distal and proximal portions 354, 356—such that the core 331 is threaded through the mounting structure 350. However, molding the mounting structure 350 around the core 331 has advantages from a manufacturing perspective due to the ability to automate the procedure, ensure good coupling between the mounting structure 350 and the core 331, avoid the need to thread an extremely small core 331 through an essentially equally small opening, prevents gaps and misfits between the core 331 and the mounting structure 350 that could lead to poor handling and/or damage to the sensing components, and other factors.

The various features of the mounting structure 300 (e.g., sidewall shapes, recess/opening sizes, etc.) can be precisely defined to match those of the sensing element, core, coils, communication lines, and/or other components that are used in conjunction with the mounting structure. This increased precision of the mounting structure 300 relative to the components that it will be used with allows for the structural support required to limit the transfer of external forces (e.g., from curvature of the intravascular device passing through a vessel) to the sensing element, which can cause errors in the resulting measurements of the sensing element, to be achieved through a minimum sized mounting structure. Further, as a result of the reduced length of the mounting structures of the present disclosure compared to those of currently available devices, which is about 0.093″ in some instances, the overall flexibility of the distal portion of the intravascular device can be increased, which leads to better maneuverability, increased accessibility, and more precise control of the intravascular device.

Referring now to FIG. 10, shown therein is the pressure sensor 306 mounted in a face down configuration using a mounting structure in accordance with the present disclosure. In that regard, the pressure sensor 306 is mounted such that the diaphragm 314 faces downwards toward the core 331.

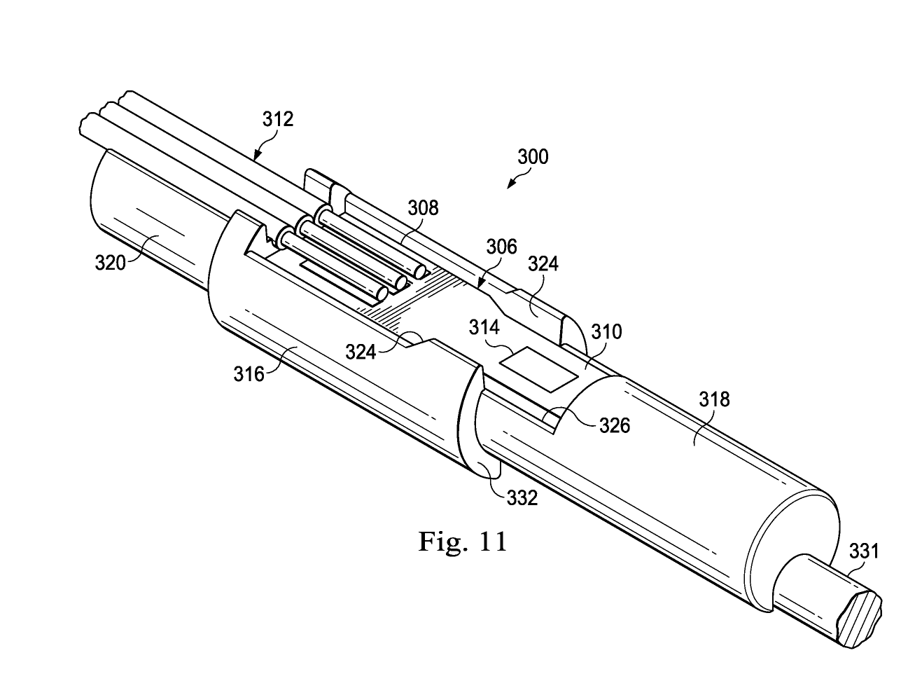

Referring now to FIG. 11, shown therein is the pressure sensor 306 mounted in a face up configuration using a mounting structure in accordance with the present disclosure. In that regard, the pressure sensor 306 is mounted such that the diaphragm 314 faces upwards away from the core 331.

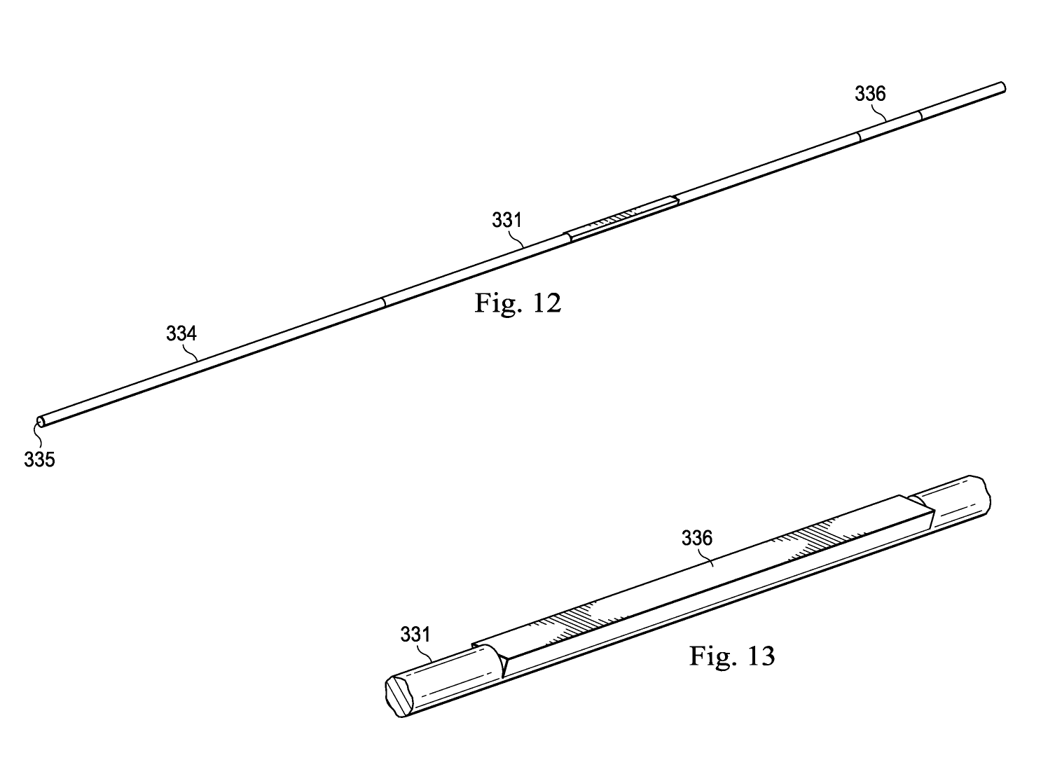

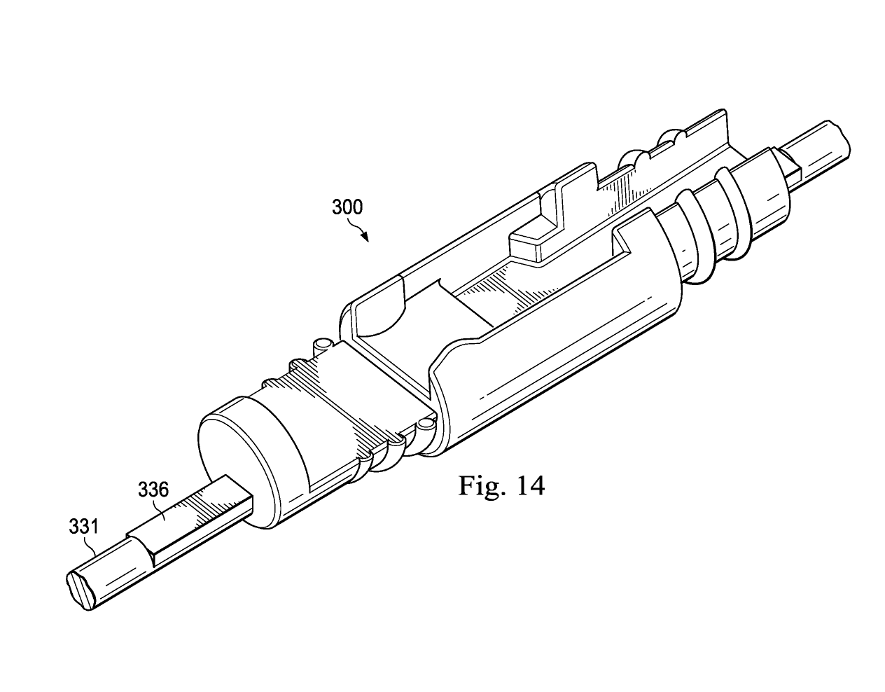

Referring now to FIGS. 12-23, shown therein are aspects of assembling a distal portion of a guide wire according to an embodiment of the present disclosure. Referring initially to FIG. 12, shown therein is a distal most portion of a core wire 331. As shown, the core wire 331 includes a section 334 extending to a distal tip 335 of the core wire and a section 336 spaced from the distal tip 335 by approximately 3 cm. Sections 334 and 336 are flattened portion of the core wire 331. In some embodiments, the sections 334 and 336 are flattened in a similar manner such that the flattened portions of each section extend in a common plane or at least in planes extending parallel to one another. However, in other embodiments the flattened portion of section 336 extends in a plane that as at an oblique or right angle with respect to the flattened portion of section 334. FIG. 13 provides a more detailed view of section 336. As shown, section 336 has a length of approximately 1.9 mm in some implementations. The upper portion of section 336 is the flattened portion of the section in the embodiment of FIG. 13.

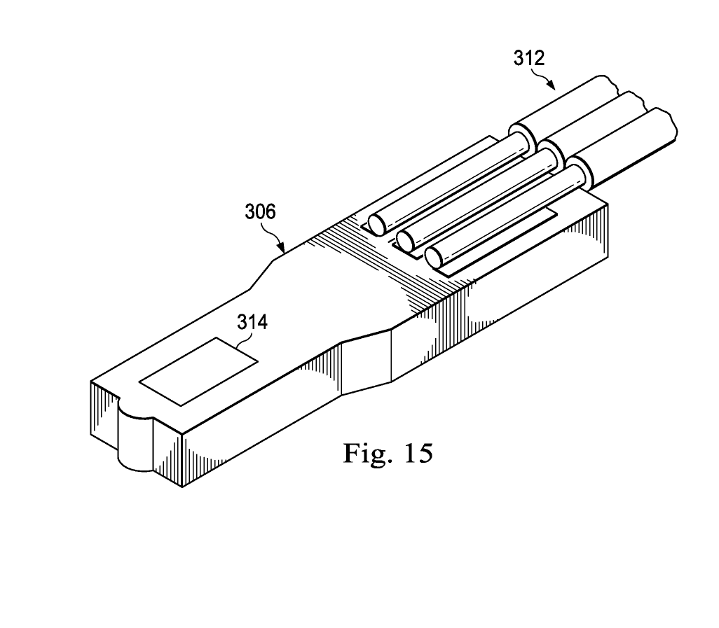

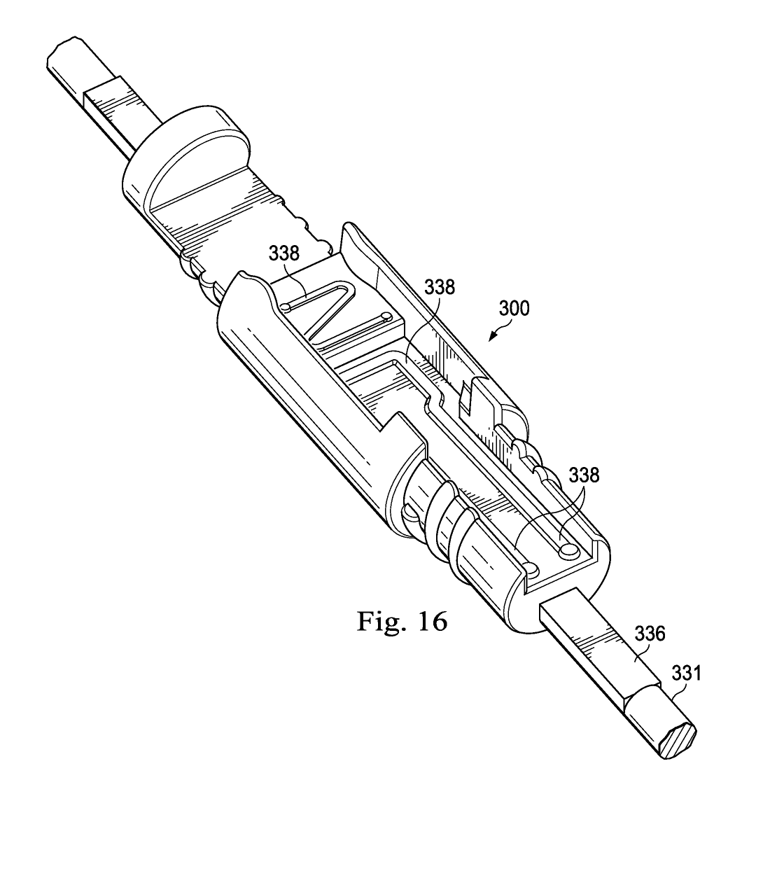

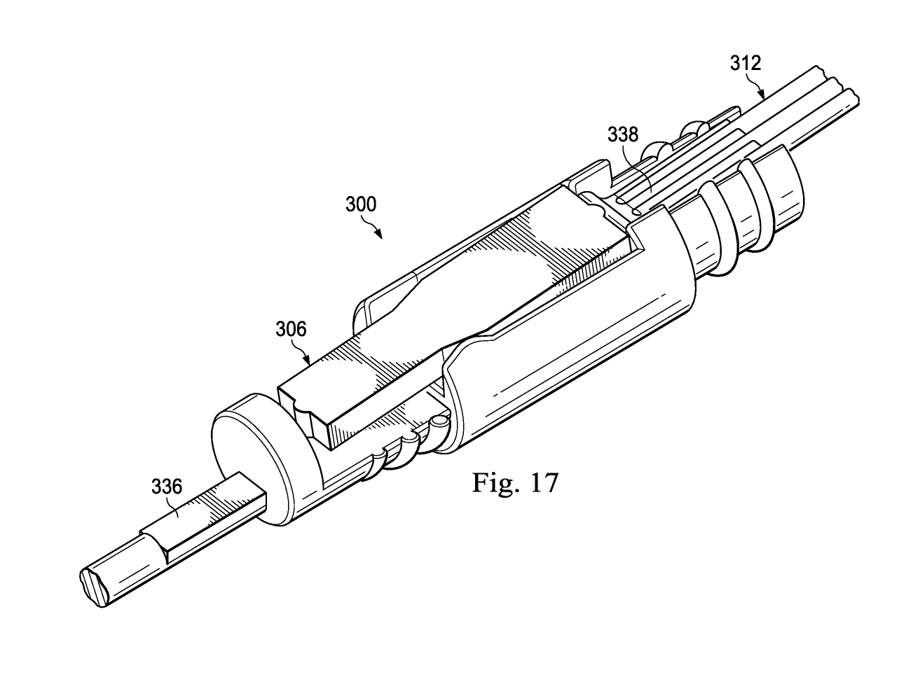

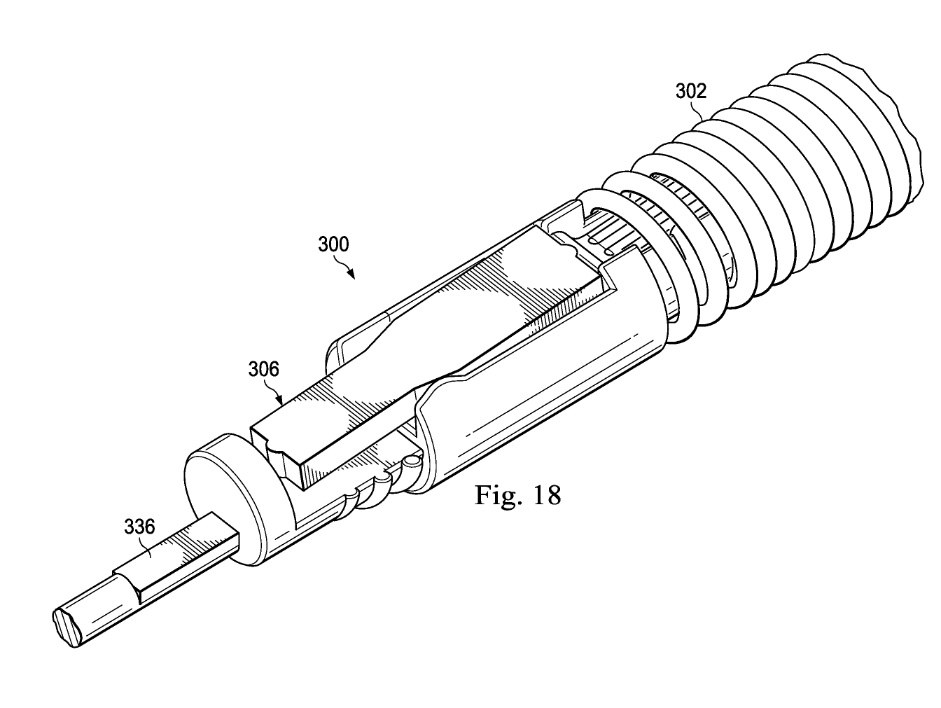

Referring now to FIG. 14, the mounting structure 300 is secured to section 336 of the core wire 331. In that regard, the mounting structure 300 may be secured to section 336 utilizing any of the techniques described above. FIG. 15 shows the pressure sensor 306 and a plurality of conductors 312, depicted as a trifilar, electrically coupled to the pressure sensor 306. FIG. 16 shows an adhesive 338 being applied to surfaces of the mounting structure 300. In the illustrated embodiment, the adhesive 338 is applied to the inner surfaces of the mounting structure 300 where the pressure sensor 306 and conductors 312 are to be secured. In that regard, FIG. 17 shows the pressure sensor 306 mounted in a face down configuration. As shown, the adhesive 338 applied to surface secures the pressure sensor 306 and the conductors 312 to the mounting structure 300, including surrounding portions of the pressure sensor 306 and/or the conductors 312 in some instances.

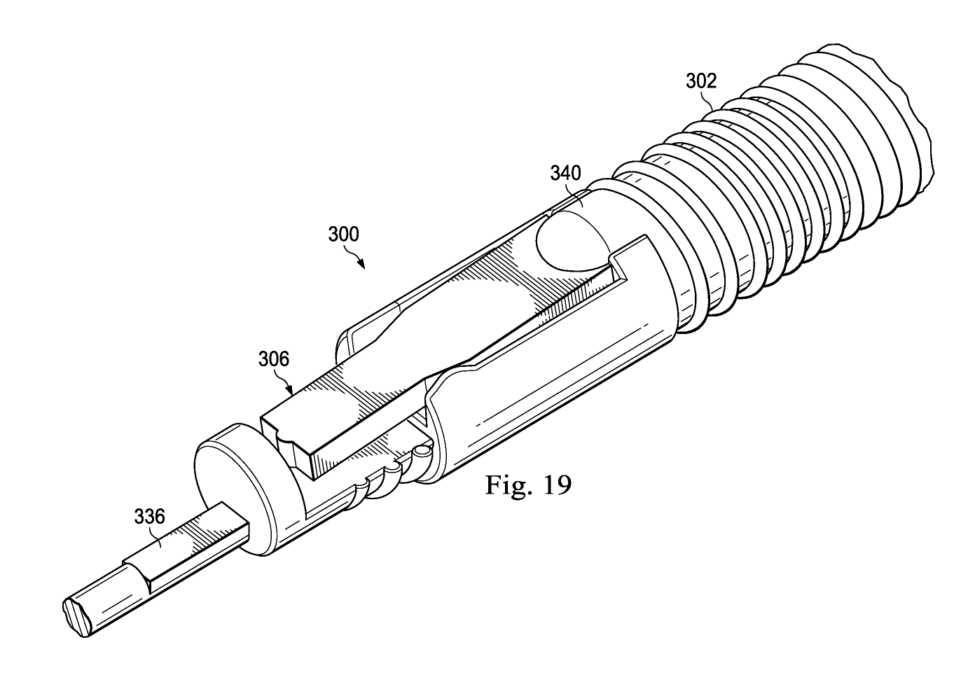

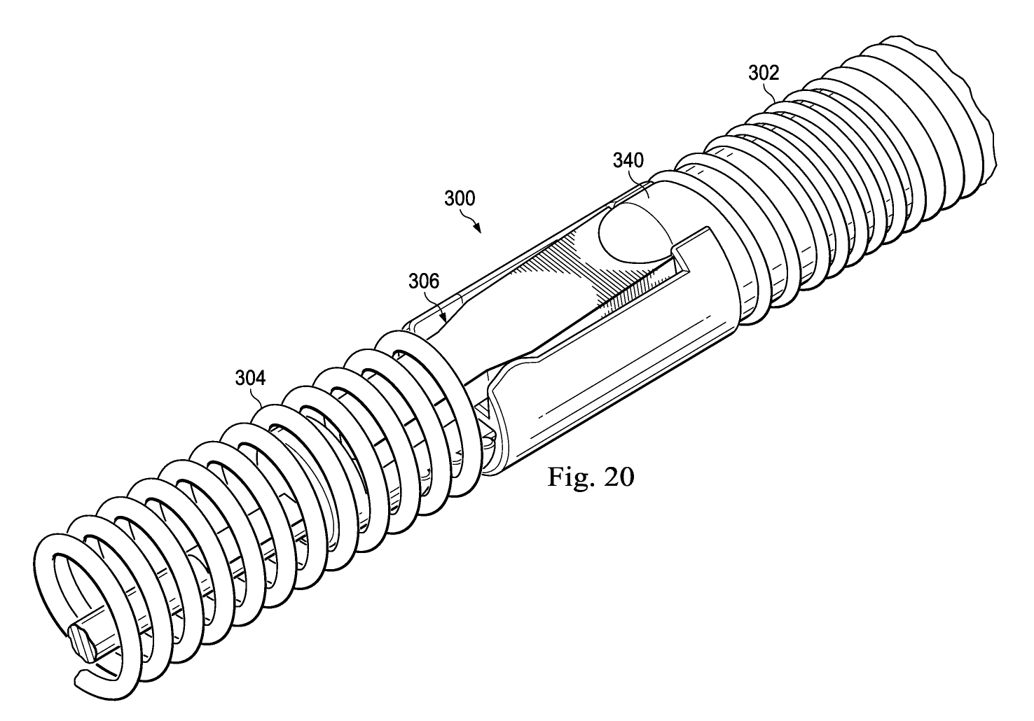

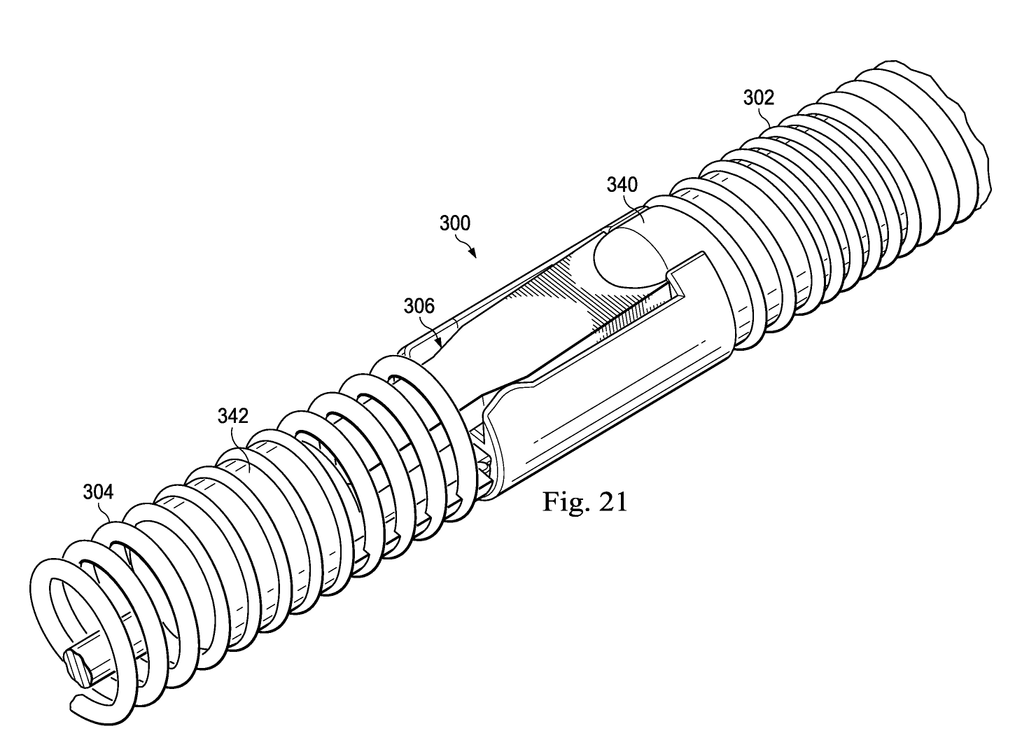

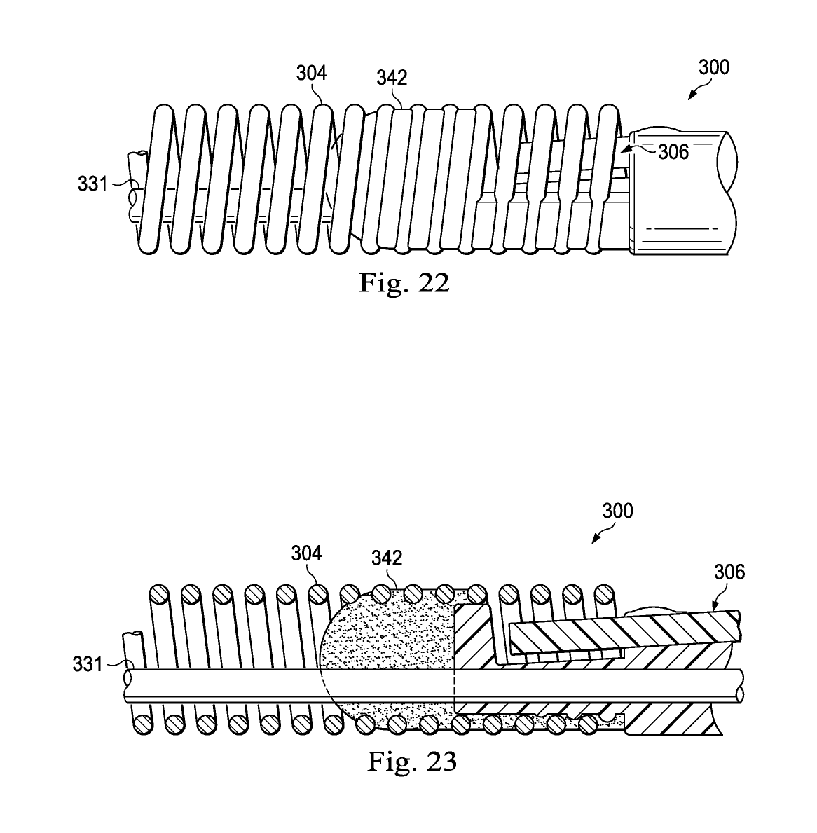

As shown in FIG. 18, with the pressure sensor 306 mounted to the mounting structure the proximal coil 302 is positioned adjacent to a proximal end portion of the mounting structure 300. FIG. 19 shows the proximal coil 302 being secured to the proximal end portion of the mounting structure 300 with an adhesive 340. FIG. 20 shows the distal coil 304 being positioned adjacent to a distal end portion of the mounting structure 300. FIG. 21 shows the distal coil 304 being secured to the distal end portion of the mounting structure 300 with an adhesive 342. In some instances, one or both of the adhesives 340, 342 are cured using one or more of heat, light, and/or other energy sources. In that regard, it is understood that the coils 302, 304 may be put on in any order and that the adhesives 340, 342 may be cured simultaneously and/or individually. To that end, it is understood that one of the adhesives 340, 342 is cured prior to putting the other coil 304, 302, respectively, onto the assembly in some instances. FIG. 22 provides a side view of the distal portion of the intravascular device, including the distal coil 304 secured to the mounting structure 300. FIG. 23 is similar to FIG. 22, but provides a cross-sectional side view of the distal portion of the intravascular device. As shown, a section of the distal coil 304 extends over a pressure sensitive region of the pressure sensing component containing the diaphragm 314.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A guide wire, comprising:
a first flexible element;
a second flexible element;
a mounting structure coupled to the first and second flexible elements such that a central portion of the mounting structure separates the first flexible element from the second flexible element, the mounting structure comprising a recess within an outer surface, the recess sized and shaped to receive a pressure sensing component;
a pressure sensing component mounted within the recess of the mounting structure;
a core extending along a length of the mounting structure such that a first portion of the core is positioned within the first flexible element and a second portion of the core is positioned within the second flexible element; and
at least one conductor having a proximal section and a distal section, wherein the distal section of the at least one conductor is coupled to the pressure sensing component and the proximal section of the at least one conductor is coupled to at least one connector;
wherein the first flexible element, the second flexible element, and the mounting structure each have an outer diameter of 0.018″ or less.

2. The guide wire of claim 1, wherein the mounting structure further comprises an opening extending along its length, wherein the core is positioned within the opening.

3. The guide wire of claim 2, wherein a first portion of the mounting structure and a first portion of the core define a first alignment feature sized and shaped to align engagement of the first flexible element with the mounting structure.

4. The guide wire of claim 3, wherein the first alignment feature has a circular cross-sectional profile.

5. The guide wire of claim 4, wherein a section of an outer surface of the first portion of the core defines at least a portion of the circular cross-sectional profile of the first alignment feature.

6. The guide wire of claim 4, wherein the first alignment feature has a cross-sectional diameter less than a cross-sectional diameter of the central portion of the mounting structure.

7. The guide wire of claim 5, wherein a second portion of the mounting structure and a second portion of the core define a second alignment feature sized and shaped to align engagement of the second flexible element with the mounting structure.

8. The guide wire of claim 7, wherein the second alignment feature has a circular cross-sectional profile.

9. The guide wire of claim 6, wherein a section of an outer surface of the first portion of the core defines at least a portion of the circular cross-sectional profile of the second alignment feature.

10. The guide wire of claim 3, wherein the central portion of the mounting structure includes the recess.

11. The guide wire of claim 2, wherein the opening is sized and shaped such that the core received within the opening is coaxial with respect to a central longitudinal axis of the mounting structure.

12. The guide wire of claim 2, wherein the opening is sized and shaped such that the core received within the opening is radially offset with respect to a central longitudinal axis of the mounting structure.

13. The guide wire of claim 12, wherein the opening is radially offset in a direction away from the recess of the mounting structure.

14. The guide wire of claim 2, wherein the mounting structure is formed of a conductive material.

15. The guide wire of claim 14, wherein the core is fixedly secured to the mounting structure with solder.

16. The guide wire of claim 2, wherein the mounting structure is formed of a non-conductive material.

17. The guide wire of claim 16, wherein the core is fixedly secured to the mounting structure with an adhesive.

18. The guide wire of claim 2, wherein the opening of the mounting structure is spaced from outer surfaces of the mounting structure such that mounting structure surrounds the core positioned within the opening.

19. The guide wire of claim 18, wherein the mounting structure is molded around the core.

20. The guide wire of claim 1, wherein the mounting structure includes at least one structural feature adjacent to the recess for mating with at least one corresponding structural feature of the pressure sensing component.

21. The guide wire of claim 20, wherein the at least one structural feature of the mounting structure is a projection and the at least one structural feature of the pressure sensing component is a recess.

22. A method of assembling a guide wire, the method comprising:
providing a core wire with a flattened section;
securing a mounting structure to the flattened section of the core wire, the mounting structure comprising a recess within an outer surface, the recess sized and shaped to receive a pressure sensing component;
securing a pressure sensing component within the recess of the mounting structure, the pressure sensing component electrically coupled to a plurality of conductors;
securing a first flexible element to a proximal portion of the mounting structure;
securing a second flexible element to a distal portion of the mounting structure such that a section of the second flexible element extends over a pressure sensitive region of the pressure sensing component; and
electrically coupling the plurality of conductors to a connector adjacent a proximal portion of the core wire.

23. The method of claim 22, wherein a first portion of the mounting structure and a first portion of the core wire define a first alignment feature sized and shaped to align engagement of the first flexible element with the mounting structure.

24. The method of claim 23, wherein the first alignment feature has a cross-sectional diameter less than a cross-sectional diameter of a central portion of the mounting structure.

25. The method of claim 23, wherein a second portion of the mounting structure and a second portion of the core wire define a second alignment feature sized and shaped to align engagement of the second flexible element with the mounting structure.

26. The method of claim 22, wherein the mounting structure further comprises an opening extending along its length, wherein the core wire is positioned within the opening.

27. The method of claim 26, wherein the opening is sized and shaped such that the core received within the opening is coaxial with respect to a central longitudinal axis of the mounting structure.

28. The method of claim 26, wherein the opening is sized and shaped such that the core received within the opening is radially offset with respect to a central longitudinal axis of the mounting structure.

29. The method of claim 28, wherein the opening is radially offset in a direction away from a recess of the mounting structure.

30. The method of claim 22, wherein securing the mounting structure to the flattened section of the core wire includes molding the mounting structure around the core wire.
